(19) United States
(12) Patent Application Publication
(10) Pub. No.: US 2012/0208230 A1
(43) Pub. Date: Aug. 16, 2012

(54) Em-Lacquer Water Solution Mixed with Natural Component, Method for Preparing Fermented Anti-Oxidant Material Using the Same, and Method for Processing the Material

(76) Inventor: Jeong Wan Ha, Seoul (KR)

(21) Appl. No.: 13/372,333

(22) Filed: Feb. 13, 2012

(30) Foreign Application Priority Data
Feb. 15, 2011 (KR) 1020110013137

(51) Int. Cl. C12P 39/00; C12N 1/20; C12N 1/14; C12N 1/16

(52) U.S. Cl. 435/42; 435/255.1; 435/252.9; 435/254.1; 435/252.1; 435/255.4; 435/252.5

(57) ABSTRACT

The present invention relates to an EM-lacquer water solution mixed with a natural component, a method for preparing a fermented anti-oxidant material using the same, and a method for processing the material. Particularly, the EM-lacquer water solution mixed with a natural component is prepared by mixing an EM-lacquer solution, which is a mixture of a crude solution of effective micro-organisms (EM) 20˜35 weight percent and a lacquer water solution 65˜80 weight percent, and at least one natural component selected from a group comprising of organic brown sugar, bay salt, mandarin orange oil, Japanese apricot concentrate, Opuntia humifusa powder, dried orange peel powder, green tea powder, sea tangle powder, rice bran, camellia oil and bean powder, on the basis of the EM-lacquer water solution 20 L. Therefore, it can be used for daily life, industry, environmental purification and the like because it is made of non-toxic materials.

### CLAIM TO FOREIGN PRIORITY

This application claims priority to the filing date of Korean Patent Application No. 10-2011-0013137, filed Feb. 15, 2011, the contents of all of which are hereby incorporated by reference.

### FIELD OF THE INVENTION

The present disclosure relates to an EM-lacquer water solution mixed with a natural component, a method for preparing a fermented anti-oxidant material using the same, and a method for processing the material. More particularly, it relates to an EM-lacquer water solution mixed with a natural component to prepare a material, which can actively use active ingredients, anti-oxidant properties and anti-oxidant advantages of micro-organisms generated by the fermentation, and to use in daily life, industry, for environmental purification and the like; a method for preparing a fermented anti-oxidant material using the same; and a method for processing the material.

### BACKGROUND OF THE INVENTION

In general, oxygen is converted to various reactive oxygen molecules by many physical, chemical and environmental causes. When the reactive oxygen molecules are accumulated in a human body due to a problem with the anti-oxidant defense wall, oxidative stress is induced, and it damages cell components and thus, causes various diseases such as aging, stroke and the like.

Studies about the anti-oxidant began as being interested in the reactive oxygen generation in vivo, the toxicity, defense and elimination mechanisms in vivo and the like with the discovery of a super oxide dismutase (SOD), an enzyme eliminating superoxide radicals, of McCord and Fridovich on 1969.

The study to develop the anti-oxidant as a food additive is being converted to a study to find an anti-oxidant as a drug for anti-aging and treating diseases, as was recently identified the fact that the reactive oxygen molecules act as a direct cause of various diseases, aging and the like.

The anti-oxidants, developed and used up to now, are synthetic anti-oxidants such as tert-butylhydroxytoluene (BHT), tert-butylhydroxyanisol (BHA) and the like, natural anti-oxidants such as α-tocopherol, vitamin C, carotenoids, flavonoids, tannin and the like, and anti-oxidant enzymes such as SOD. However, the application of these anti-oxidants is restricted due to their toxicity, low-activity and limited use.

Thus, studies to obtain safer and stronger anti-oxidants from natural materials or micro-organism metabolites are actively going on.

A objective of the anti-oxidant is spreading from a lipid peroxidation inhibitor to a preventive anti-oxidant material inhibiting the self-generation of free radicals and reactive oxygen, which become direct causes of oxidative damages in vivo, and the direction of the study has been changed to high molecular weight materials such as SOD and low molecular weight natural anti-oxidants, which are very practical in synthetic anti-oxidants and stable. Further, as a defense system to protect living things from oxidative damage and various diseases caused thereby, studies for identifying the natural anti-oxidant materials, anti-oxidant defense mechanism, and gene expression and cellular regulation of anti-oxidants are in progress.

Domestically, searches for an anti-oxidant from nature started in 1992, and studies about various novel anti-oxidant materials such as metabolites of micro-organisms have begun. Now, studies directed to developing anti-inflammatory agents, neuroprotective agents, skin anti-aging agents and food additives are in progress.

### SUMMARY OF THE INVENTION

The present invention has been made according to said development. It is an objective of the present invention to provide an EM-lacquer water solution mixed with a natural component to prepare a material, which can actively use active ingredients, anti-oxidant properties and anti-oxidant advantages of micro-organisms generated by the fermentation, and to use in daily life, industry, for environmental purification and the like; a method for preparing a fermented anti-oxidant material using the same; and a method for processing the material.

The EM-lacquer water solution mixed with a natural component comprises an EM-lacquer water solution, which is a mixture of a crude solution of effective micro-organisms (EM) 20˜35 weight percent and a lacquer water solution 65˜80 weight percent, and at least one natural component selected from a group comprising organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters (L).

In order to accomplish said purpose, the method for preparing a fermented anti-oxidant material using an EM-lacquer water solution mixed with a natural component of the present invention is characterized by comprising the steps of preparing an EM-lacquer water solution by mixing a crude solution of effective micro-organisms (EM) 20˜35 weight percent and a lacquer water solution 65˜80 weight percent, preparing an EM-lacquer water solution mixed with a natural component by mixing the EM-lacquer water solution, and at least one selected from a group consisting of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 L; and fermenting and aging the EM-lacquer water solution mixed with a natural component at room temperature of 15˜40° C. for 2 months.

At this time, the step of fermenting and aging at room temperature comprises the steps of fermenting at 38˜40° C. for 10 days to activate micro-organisms, and aging by fermenting at 17˜27° C. for 50 days after the fermenting step to stabilize the micro-organisms.

The method further comprises a step of removing impurities using a filter after the step of fermenting and aging at room temperature.

At this time, the filter is any one of a 10 μm bag filter and a 0.2 μm micro-filter.

In order to accomplish said purpose, the method for processing a fermented anti-oxidant material using an EM-lacquer water solution mixed with a natural component of the present invention is characterized by processing the fermented anti-oxidant material using an EM-lacquer water solution mixed with a natural component (which is prepared by the steps of preparing an EM-lacquer water solution by mixing a crude solution of effective micro-organisms (EM) 20˜35 weight percent and a lacquer water solution 65˜80 weight percent, preparing an EM-lacquer water solution mixed with a natural component by mixing the EM-lacquer water solution, and at least one of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters, fermenting and aging the EM-lacquer water solution mixed with a natural component at room temperature of 15˜40° C. for 2 months, and processing into a powder at a vacuum pressure of 1.34×10⁻⁶ kg/cm² to 1.34×10⁻³ kg/cm² and a temperature of 0˜55° C. using a freeze dryer.

The fermented anti-oxidant material using the EM-lacquer water solution mixed with a natural component can be processed into a concentrated solution at the pressure of 2˜8 kg/cm² and a temperature of 5˜55° C. using a food concentrator.

Further, the fermented anti-oxidant material using the EM-lacquer water solution mixed with a natural component can be processed into a concentrated solution at a vacuum pressure of 1.36×10⁻⁴ kg/cm² to 1.03 kg/cm² and a temperature of 0˜80° C. using a low temperature vacuum concentrator.

At this time, the fermented anti-oxidant material using the EM-lacquer water solution mixed with a natural component can be processed into a distilled solution at a vacuum pressure of 1.36×10⁻⁴ kg/cm² to 1.03 kg/cm² and a temperature of 0˜80° C. using a low temperature vacuum distiller.

The fermented anti-oxidant material using the EM-lacquer water solution mixed with a natural component can be processed into a distilled solution at a vacuum pressure of 1.34×10⁻⁶ kg/cm² to 1.34×10⁻³ kg/cm² and a temperature of 60˜150° C. using a high temperature vacuum distiller.

Further, the fermented anti-oxidant material using the EM-lacquer water solution mixed with a natural component can be processed into a distilled solution at the pressure of 1˜3 kg/cm² and the temperature of 110˜120° C. using an indirect heating distiller.

Alternatively, the present invention is characterized by further mixing any one of citric acid and lactic acid with the fermented anti-oxidant material to control the potential of hydrogen (pH) so as to obtain a pH fermented anti-oxidant material.

At this time, the citric acid is mixed with the fermented anti-oxidant material in the amount of 20 weight percent or less of the fermented anti-oxidant material, the lactic acid is mixed with the fermented anti-oxidant material in the amount of 80 weight percent or less of the fermented anti-oxidant material, and both of the citric acid and the lactic acid are mixed with the fermented anti-oxidant material in the amount of 10 weight percent or less and 70 weight percent or less of the fermented anti-oxidant material, respectively.

Further, the present invention is characterized by further mixing any one of agar powder and konjak powder with the pH fermented anti-oxidant material in the amount of 10 weight percent or more of the pH fermented anti-oxidant material, and heating the mixture to the temperature of 60° C. or more for dissolving, making to a viscous water solution, and drying at room temperature of 5° C. or more.

### DETAILED DESCRIPTION

Hereinafter, preferred embodiments of the present invention will be described in detail enough to be easily accomplished by a person skilled in the art.

The EM-lacquer water solution mixed with a natural component of the present invention is prepared by mixing an EM-lacquer water solution, which is a mixture of a crude solution of effective micro-organisms (EM) 20˜35 weight percent and a lacquer water solution 65˜80 weight percent, and at least one natural component selected from a group consisting of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on the basis of the EM-lacquer water solution 20 liters (L).

The effective micro-organisms (EM) refer to a blend and a culture of several tens of micro-organisms beneficial to people. Micro-organisms such as yeast, lactobacillus, fermenting fungi, photosynthetic bacteria and the like are known to have outstanding effects on odor removal, water purification, food fermentation and the like.

In the present invention, generally, five seed cultures are selected among EM, which are mainly used for livestock and agriculture in the market. Seed cultures containing each seed material are purchased, respectively and equally mixed, or the pre-mixed seed cultures are purchased.

The five seed culture used herein are seed cultures comprising 2.9×10⁴ cfu/ml or more of Rhodopseumdomomas Palustris, 1.3×10⁴ cfu/ml or more of Rhodopseumdomomas Spheroies, 2.7×10⁴ cfu/ml or more of Candida Valida, 2.4×10⁴ cfu/ml or more of lactobacillus paracasei and 1.2×10⁴ cfu/ml or more of bacillus subtilis, respectively.

Each seed contained in the five seed culture is the micro-organisms cultured under the following conditions.

Namely, the Rhodopseumdomomas Palustris and Rhodopseumdomomas Spheroies are cultured at 31° C.±1° C. under anaerobic conditions for 7 days, and have a brown color and a pH of 9; Candida Valida is cultured at 33° C.±1° C. under anaerobic conditions for 5 days, and has a milk color and a pH of 7; lactobacillus paracasei is cultured at 32° C.±1° C. under anaerobic conditions for 5 days, and has a brown color and has a pH of 4; and bacillus subtilis is cultured at 38° C.±1° C. under aerobic conditions (inserting air passed through a sterilizing filter) for 10 days, and has a pale yellow color and a pH of 6.8˜7.0.

Herein, the crude EM solution used in the present invention comprises a liquid nutritional supplement containing plural nutrients required for culture 1˜2 kg, treacle 1˜2 kg, seed culture containing said five seeds 0.5˜2 kg, bay salt 0.1˜1 kg and sterilized yellow soil 0.1˜1 kg.

The lacquer water solution is a crude solution prepared by heating a lacquer tree in a pot for a long time and extracting thereof, and the lacquer solution is prepared by various methods, for example, a method of drying a lacquer tree for a long time and then adding soju followed by heating; a method of adding the equivalent amount of leaves of a chestnut tree, and a method for preparing a lacquer solution removing urushiol, the material of the lacquer tree which causes itching. Accordingly, in the present invention, edible true lacquer solution is used as the lacquer solution composing the lacquer water solution. More specifically, a concentrated liquid product comprising a solid of lacquer solution 0.4˜0.45%, which does not contain urushiol causing an allergy in the human body, is used, and the lacquer water solution is completely prepared by mixing the concentrated liquid lacquer solution and water to the weight ratio of 1:20˜40.

The processes for manufacturing the EM-lacquer water solution mixed with natural components according to the present invention comprising said components will be described as follows.

First, after mixing the crude EM solution and the lacquer water solution, the mixture is initially fermented at 38˜40° C. for 10 days, and then fermented at 17˜20° C. for 20 days to increase the population of the micro-organism. After completing the fermentation, the mixture is purified using a filter to form a liquid not containing impurities. The EM-lacquer water solution mixed with natural components according to the present invention is accomplished.

The method for preparing a fermented anti-oxidant material using an EM-lacquer water solution mixed with a natural component according to the present invention comprises the steps of preparing an EM-lacquer water solution by mixing a crude solution of effective micro-organisms (EM) 20˜35 weight percent and a lacquer water solution 65˜80 weight percent, preparing an EM-lacquer water solution mixed with a natural component by mixing the EM-lacquer water solution, and at least one selected from a group consisting of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on the basis of the EM-lacquer water solution 20 L; and fermenting and aging the EM-lacquer water solution mixed with a natural component at room temperature of 15˜40° C. for 2 months.

More specifically, the lacquer water solution is put into a tank and sterilized by heating to 60˜70° C., and when the sterilized lacquer water solution is cooled to 50° C. or less, the crude EM solution is added thereto. Then, when the temperature comes to 35° C. or less, at least one component selected from a group consisting of organic brown sugar, bay salt, mandarin orange oil, Japanese apricot concentrate, Opuntia humifusa powder, dried orange peel powder, green tea powder, sea tangle powder, rice bran, camellia oil and bean powder is put into a tank and the culture is started.

Then, the optimum temperature and time for fermenting and aging is: after conducting the initial fermenting step to activate the micro-organism at 38° C.˜40° C. for 10 days, secondly the aging step to stabilize the micro-organism at about 17˜20° C. for 50 days is conducted, and thus, the fermenting and aging steps of about 2 months are completed.

Herein, the step of fermenting and aging at room temperature is divided into two steps in detail.

The first step is to ferment the prepared EM-lacquer water solution mixed with a natural component at 38˜40° C. for 10 days to activate the micro-organisms.

The second step is to age by fermenting the micro-organisms activated through the first fermenting step at 17˜27° C. for 50 days to stabilize the micro-organisms.

After completing the step of fermenting and aging at room temperature, a step removing impurities using a filter is further conducted, and the filter is any one selected from a 10 μm bag filter and a 0.2 μm micro-filter.

Alternatively, the EM-lacquer water solution mixed with a natural component can be aged by fermenting at low temperature of 17˜20° C. for 60 days, not through the said first and second steps.

Because the fermented anti-oxidant material using an EM-lacquer water solution mixed with a natural component prepared by the method described above can increase the culture population of EM and improve the growth condition and metabolic function of EM, the fermented material can largely increase nutrients and anti-oxidant materials generated during a metabolic process of a micro-organism, and it is easy to process because it is more diluted and less sticky than a crude EM solution.

This fermented anti-oxidant material can be used for: 1. raw materials of preservatives and anti-oxidants, 2. a raw material of nutrients (plant and animal nutrients), 3. raw materials of sterilizers and anti-microbial agents, 4. a lubricant and an additive (anti-corruption and maintaining anti-corrosion during metal processing), 5. a deodorizer (bad smell removal), 6. a natural detergent additive (raw materials of surfactant, fabric softener and low-toxic food machinery cleaner), 7. an oil decomposition agent (stimulating the biodegradation of oils contaminating the soil and sea), 8. a raw material of natural anti-corrosion agents (possible to be used as a raw material of anti-corrosion agents of metal: rust removal and prevention), 9. a raw material of oil-water separators, 10. non-toxic scale and rust removers, 11. supplement additives of detergents and cleaners, 12. a water-treatment enhancer (purification) of lakes, rivers, ponds and the like, 13. cosmetic cleanser additive and nutrient, and the like.

In the method for processing a fermented anti-oxidant material prepared by fermenting an EM-lacquer water solution mixed with a natural component according to the present invention, the fermented anti-oxidant material using an EM-lacquer water solution mixed with a natural component (which is prepared by the steps of preparing an EM-lacquer water solution by mixing a crude solution of effective micro-organisms (EM) 20˜35 weight percent and a lacquer water solution 65˜80 weight percent, preparing an EM-lacquer water solution mixed with a natural component by mixing the EM-lacquer water solution, and at least one of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on the basis of the EM-lacquer water solution 20 L; and fermenting and aging the EM-lacquer water solution mixed with a natural component at room temperature of 15˜40° C. for 2 months) is processed into the form of a powder by freeze drying at the vacuum pressure of 10⁻³˜1 torr (which is approximately 1.34×10⁻⁶ kg/cm² to 1.34×10⁻³ kg/cm² and which is approximately 1.93×10⁻⁵ pounds per square inch (psi) to 0.019 psi) and the temperature of 0˜−55° C. using a freeze dryer followed by pulverizing thereof.

By preparing the material into the form of a powder using the freeze dryer, it is expected to make long-term storage easy, reduce delivery costs and prevent damage during storage and delivery.

The fermented anti-oxidant material using the EM-lacquer water solution mixed with a natural component can be processed using a food concentrator instead of the freeze dryer. At this time, it is processed into the form of a concentrated solution by concentrating at the pressure of 2˜8 kg/cm² (which is approximately 28.45 psi to 113.79 psi) and the temperature of 5˜55° C.

Further, the fermented anti-oxidant material using the EM-lacquer water solution mixed with a natural component can be processed using a low temperature vacuum concentrator instead of the freeze dryer or the food concentrator. At this time, it is processed into the form of a concentrated solution by concentrating at the vacuum pressure of 10⁻¹˜760 torr (which is approximately 1.36×10⁻⁴ kg/cm² to 1.03 kg/cm² and which is approximately 1.93×10⁻³ psi to 14.70 psi) and the temperature of 0˜80° C.

Further, the fermented anti-oxidant material using the EM-lacquer water solution mixed with a natural component can be processed using a low temperature vacuum distiller instead of the freeze dryer, the food concentrator or the low temperature vacuum concentrator. At this time, it is processed into the form of a distilled solution by distilling at the vacuum pressure of 10⁻⁴˜760 torr (which is approximately 1.36×10⁻⁴ kg/cm² to 1.03 kg/cm² and which is approximately 1.93×10⁻³ psi to 14.70 psi) and the low temperature of 0˜80° C.

Further, the fermented anti-oxidant material using the EM-lacquer water solution mixed with a natural component can be processed using a high temperature vacuum distiller instead of the freeze dryer, the food concentrator, the low temperature vacuum concentrator or a low temperature vacuum distiller. At this time, it is processed into the form of a distilled solution by distilling at the vacuum pressure of 10⁻³˜1 torr (which is approximately 1.34×10⁻⁶ kg/cm² to 1.34×10⁻³ kg/cm² and which is approximately 1.93×10⁻⁵ psi to 0.019 psi) and the high temperature of 60˜150° C.

Further, the fermented anti-oxidant material using the EM-lacquer water solution mixed with a natural component can be processed using an indirect heating distiller instead of the freeze dryer, the food concentrator, the low temperature vacuum concentrator, the low temperature vacuum distiller or the high temperature vacuum distiller. At this time, it is processed into the form of a distilled solution by indirectly heating and distilling at the pressure of 1˜3 kg/cm² (which is approximately 14.22 psi to 46.67 psi) and the temperature of 110˜120° C.

Alternatively, any one of citric acid and lactic acid can be further mixed with the fermented anti-oxidant material processed with the various apparatuses to control the potential of hydrogen (pH) so as to obtain a pH fermented anti-oxidant material.

At this time, the citric acid is mixed with the fermented anti-oxidant material in the amount of 20 weight percent or less of the fermented anti-oxidant material, the lactic acid is mixed with the fermented anti-oxidant material in the amount of 80 weight percent or less of the fermented anti-oxidant material, and both of the citric acid and the lactic acid are mixed with the fermented anti-oxidant material in the amount of 10 weight percent or less and 70 weight percent or less of the fermented anti-oxidant material, respectively.

The citric acid has strong sterilization and odor removal functions, and the lactic acid has strong scale removal, rust removal, anti-corrosion and oil-water separation functions, and therefore, it can further improve the efficacy of the pH fermented anti-oxidant material.

Herein, any one of agar powder and konjak powder can be further mixed with the pH fermented anti-oxidant material in the amount of 10 weight percent or more of the pH fermented anti-oxidant material, and the mixture can be heated to the temperature of 60° C. or more for dissolving, to make a viscous water solution and dried at room temperature of 5° C. or more.

The agar powder and the konjak powder have characteristics that they start to slowly melt at the temperature of 60° C. or more and become liquefied, and then become solidified into the form of a jelly or curd when they cool down. The material once solidified has an advantage that it is not modified by temperature change during packaging and delivery processes because it is not modified at the temperature between room temperature and 60° C.

According to the inventive EM-lacquer water solution mixed with a natural component, the method for manufacturing the fermented anti-oxidant material thereof, and the method for processing the fermented anti-oxidant material, a material, which can actively utilize the effective ingredients, anti-oxidant properties and anti-oxidant advantages of the micro-organisms generated from the fermentation, can be made by making the EM-lacquer water solution mixed with a natural component and processing thereof using various methods, and the material can be used for daily life, industry, environmental purification and the like.

Because it is made of non-toxic materials, it can be easily applied in said various fields, and is easy to use.

Further, it is also easy to package, store and deliver.

While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

What is claimed:

1. An EM-lacquer water solution mixed with a natural component, comprising:
an EM-lacquer water solution, which is a mixture of a crude solution of effective micro-organisms (EM) 20˜35 weight percent and a lacquer water solution 65˜80 weight percent; and
at least one natural component selected from a group comprising organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters.

2. A method for preparing a fermented anti-oxidant material using an EM-lacquer water solution mixed with a natural component comprising the following steps of:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one selected from a group comprising organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters; and
fermenting and aging the EM-lacquer water solution mixed with a natural component at room temperature of 15˜40° C. for 2 months.

3. The method for preparing a fermented anti-oxidant material using an EM-lacquer water solution mixed with a natural component of claim 2, wherein the step of fermenting and aging at room temperature comprises the following steps of:
fermenting at 38˜40° C. for 10 days to activate micro-organisms; and
aging by fermenting at 17˜27° C. for 50 days after the fermenting step to stabilize the micro-organisms.

4. The method for preparing a fermented anti-oxidant material using an EM-lacquer water solution mixed with a natural component of claim 2, which further comprises a step of removing impurities using a filter after the step of fermenting and aging at room temperature.

5. The method for preparing a fermented anti-oxidant material using an EM-lacquer water solution mixed with a natural component of claim 4, wherein the filter is any one of a 10 μm bag filter and a 0.2 μm micro-filter.

6. The method for preparing a fermented anti-oxidant material using an EM-lacquer water solution mixed with a natural component of claim 2, further comprising mixing any one of a citric acid and a lactic acid with the fermented anti-oxidant material to control a potential of hydrogen (pH) so as to obtain a pH fermented anti-oxidant material.

7. The method for preparing a fermented anti-oxidant material using an EM-lacquer water solution mixed with a natural component of claim 6, further comprising mixing the citric acid with the fermented anti-oxidant material in an amount of 20 weight percent or less of the fermented anti-oxidant material.

8. The method for preparing a fermented anti-oxidant material using an EM-lacquer water solution mixed with a natural component of claim 6, further comprising mixing the lactic acid with the fermented anti-oxidant material in an amount of 80 weight percent or less of the fermented anti-oxidant material.

9. The method for preparing a fermented anti-oxidant material using an EM-lacquer water solution mixed with a natural component of claim 6, further comprising mixing both of the citric acid and the lactic acid with the fermented anti-oxidant material in an amount of 10 weight percent or less and 70 weight percent or less of the fermented anti-oxidant material, respectively.

10. The method for preparing a fermented anti-oxidant material using an EM-lacquer water solution mixed with a natural component of claim 6, further comprising:
mixing any one of agar powder and konjak powder with the pH fermented anti-oxidant material in an amount of 10 weight percent or more of the pH fermented anti-oxidant material;
heating to a temperature of 60° C. or more for dissolving;
making to a viscous water solution; and
drying at room temperature of 5° C. or more.

11. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a powder at a vacuum pressure of 1.34×10⁻⁶ kg/cm² to 1.34×10⁻³ kg/cm² and a temperature of 0˜−55° C. using a freeze dryer.

12. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a concentrated solution at a pressure of 2˜8 kg/cm² and a temperature of 5˜55° C. using a food concentrator.

13. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a concentrated solution at a vacuum pressure of 1.36×10⁻⁴ kg/cm² to 1.03 kg/cm² and a temperature of 0˜80° C. using a low temperature vacuum concentrator.

14. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 g, bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a distilled solution at a vacuum pressure of 1.36×10⁻⁴ kg/cm² to 1.03 kg/cm² and a temperature of 0˜80° C. using a low temperature vacuum distiller.

15. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a distilled solution at a vacuum pressure of 1.34×10⁻⁶ kg/cm² to 1.34×10⁻³ and a temperature of 60˜150° C. using a high temperature vacuum distiller.

16. A method for processing a fermented anti-oxidant material prepared by fermenting an EM-lacquer water solution mixed with a natural component, comprising:
preparing an EM-lacquer water solution by mixing a crude solution of effective micro-organisms (EM) 20˜35 weight percent and a lacquer water solution 65˜80 weight percent;
preparing an EM-lacquer water solution mixed with a natural component by mixing the EM-lacquer water solution, and at least one of organic brown sugar 100˜200 grams (g), bay salt 50˜150 g, mandarin orange oil 50˜100 g, Japanese apricot concentrate 50˜100 g, Opuntia humifusa powder 50˜100 g, dried orange peel powder 50˜100 g, green tea powder 5˜30 g, sea tangle powder 5˜30 g, rice bran 5˜30 g, camellia oil 5˜30 g and bean powder 5˜30 g, on a basis of the EM-lacquer water solution 20 liters;
fermenting and aging the EM-lacquer water solution mixed with a natural component at room temperature of 15˜40° C. for 2 months; and
processing into a distilled solution at a pressure of 1˜3 kg/cm² and a temperature of 110˜120° C. using an indirect heating distiller.
